(19) United States
(12) Patent Application Publication
(10) Pub. No.: US 2025/0186217 A1
(43) Pub. Date: Jun. 12, 2025

(54) INTERVERTEBRAL DEVICES

(71) Applicant: AXIS SPINE TECHNOLOGIES LTD, Reading (GB)

(72) Inventors: Christopher REAH, Reading (GB); Jonathan ARCOS, Reading (GB); Nicholas SANDHAM, Reading (GB); Frank HEUER, Reading (GB)

(21) Appl. No.: 18/914,626

(22) Filed: Oct. 14, 2024

Related U.S. Application Data
(63) Continuation-in-part of application No. 17/294,681, filed on May 17, 2021, now Pat. No. 12,115,082.

(30) Foreign Application Priority Data
Nov. 19, 2018 (GB) 1818847.4

(51) Int. Cl. A61F 2/44; A61F 2/30

(52) U.S. Cl. CPC A61F 2/4455; A61F 2/30734; A61F 2/30749; A61F 2002/30505; A61F 2002/30772

(57) ABSTRACT

The present invention relates to an intervertebral fusion device. The intervertebral fusion device comprises at least one endplate configured to be received in an intervertebral space defined between first and second vertebrae and a core component configured to engage with the at least one endplate when the at least one endplate is in the intervertebral space and the core component is inserted into the intervertebral space. The intervertebral fusion device also comprises first and second fingers and first and second recesses. A proximal end of each of the first and second fingers is attached to the endplate at or adjacent an anterior end of the endplate that first receives the core component when the core component is inserted into the intervertebral space. Each of the first and second fingers extends in the direction of insertion of the core component towards a posterior end of the endplate and is unattached along its length except at its proximal end whereby the finger flexes along its length. The first and second fingers are spaced apart from each other in a transverse direction which is orthogonal to a direction of insertion of the core component and to a direction of separation of the first and second vertebrae. The first finger defines a first protrusion at a distal end thereof and the second finger defines a second protrusion at a distal end thereof, each protrusion protruding from the respective finger in the transverse direction. The core component defines the first and second recesses near a posterior end of the core component, the first and second recesses spaced apart from each other in the transverse direction. The core component bears against the first and second protrusions during a first stage of insertion to flex the first and second fingers in the transverse direction, and during a second stage of insertion after the first stage of insertion the first and second protrusions are received in the first and second recesses respectively and under spring bias exerted by the first and second fingers to thereby retain the core component relative to the endplate.

This application is a continuation of U.S. patent application Ser. No. 17/294,681 filed on May 17, 2021, issuing as U.S. Pat. No. 12,115,082 on Oct. 15, 2024, which is a 371 of International Application No. PCT/GB2019/053273 filed on Nov. 19, 2019, which claims priority of GB Patent Application No. 1818847.4 filed on Nov. 19, 2018. These applications are fully incorporated by reference as if fully set forth herein.

### FIELD OF THE INVENTION

The present invention relates to intervertebral devices and more specifically to intervertebral fusion devices.

### BACKGROUND TO THE INVENTION

Adjacent vertebrae in the spinal column are coupled to each other by an intervertebral disc. The intervertebral disc holds the adjacent vertebrae together and functions as a cushion between the vertebrae whilst allowing for relative movement of the vertebrae. Problems with intervertebral discs arise from one or more of a range of diseases and conditions. A surgical procedure, such as spinal fusion, may be used to address such problems. A typical spinal fusion procedure involves partial or full removal of a problematic intervertebral disc and installation of an intervertebral device in the place of the partially or fully removed intervertebral disc.

Known intervertebral devices are of varied form and function. Many known intervertebral devices are configured to provide for adjustment of height and functional spine unit angle to address differing extents of removal of an intervertebral disc, differing anatomy and spinal deformity. Furthermore, ease of assembly, installation, including reduced impaction loads during insertion, and disassembly are design objects for known intervertebral devices aside from issues of manufacturability and cost. Some known intervertebral devices are characterised by their complexity with such complexity being liable to result in compromise on ease of assembly, installation and disassembly, in compromise on long-term reliability, or in risk to the patient, such as from wear of material from the intervertebral device over time and loss of spinal correction.

The present inventors have become appreciative of shortcomings of known intervertebral devices, such as the shortcomings mentioned above. The present invention has been devised in light of the inventors' appreciation of such shortcomings. It is therefore an object for the present invention to provide an improved intervertebral device and more specifically an improved intervertebral fusion device. It is a further object for the present invention to provide an improved method of installing an intervertebral device in an intervertebral space between first and second adjacent vertebrae and more specifically an improved method of installing an intervertebral fusion device.

### STATEMENT OF INVENTION

According to a first aspect of the present invention there is provided an intervertebral fusion device, comprising:
at least one endplate configured to be received in an intervertebral space defined between first and second vertebrae;
a core component configured to engage with the at least one endplate when the at least one endplate is in the intervertebral space and the core component is inserted into the intervertebral space;
first and second fingers; and
first and second recesses, wherein
a proximal end of each of the first and second fingers is attached to the endplate at or adjacent an anterior end of the endplate that first receives the core component when the core component is inserted into the intervertebral space,
each of the first and second fingers extends in the direction of insertion of the core component towards a posterior end of the endplate and is unattached along its length except at its proximal end whereby the finger flexes along its length,
the first and second fingers are spaced apart from each other in a transverse direction which is orthogonal to a direction of insertion of the core component and to a direction of separation of the first and second vertebrae,
the first finger defines a first protrusion at a distal end thereof and the second finger defines a second protrusion at a distal end thereof, each protrusion protruding from the respective finger in the transverse direction,
the core component defines the first and second recesses near a posterior end of the core component, the first and second recesses spaced apart from each other in the transverse direction, and
the core component bears against the first and second protrusions during a first stage of insertion to flex the first and second fingers in the transverse direction, and during a second stage of insertion after the first stage of insertion the first and second protrusions are received in the first and second recesses respectively and under spring bias exerted by the first and second fingers to thereby retain the core component relative to the endplate.

The first and second recesses are near the posterior end of the core component whereby the distal ends of the first and second fingers may be near the posterior end of the endplate in view of the first and second protrusions being defined at respective distal ends of the first and second fingers. The first and second fingers may therefore extend from the anterior end of the endplate to near the posterior end of the endplate. The first and second fingers may therefore guide the core component from first reception of the core component by the anterior end of the endplate to the second stage of insertion when the first and second protrusions are received in the first and second recesses whereupon the core component is retained relative to the endplate.

The intervertebral fusion device may comprise first and second endplates and the core component may be received between the first and second endplates when the core component is inserted into the intervertebral space. Alternatively, the intervertebral fusion device may comprise first and second endplates, the first and second fingers may be attached to the first endplate, and the second endplate may be comprised in and integrally formed with the core component. The first endplate may be placed in the intervertebral space and then the core component with its integral second endplate may be inserted into the intervertebral space and brought into engagement with the first endplate.

Where the intervertebral fusion device comprises first and second endplates, the first and second fingers may be attached to the first endplate and the intervertebral fusion device may comprise third and fourth fingers and third and fourth recesses. The third and fourth fingers may be attached to the second endplate and the third and fourth recesses may be defined by the core component. The third and fourth fingers may flex in the transverse direction during the first stage of insertion of the core component between the first and second endplates. A third protrusion defined by the third finger may be received in the third recess and a fourth protrusion defined by the fourth finger may be received in the fourth recess during the second stage of insertion of the core component. The core component may thus engage with the first and second endplates when the core component is inserted between the first and second endplates with reception of the first to fourth protrusions in their respective recesses providing for retention of the core component relative to the first and second endplates.

The first endplate may be a superior endplate and may define at least one superior screw aperture near the anterior end of the superior endplate. The second endplate may be an inferior endplate and may define at least one inferior screw aperture near the anterior end of the inferior endplate. Each at least one superior screw aperture may be at a different location in the transverse direction from each least one inferior screw aperture when the superior and inferior endplates are in registration. Each at least one superior screw aperture and each at least one inferior screw aperture may be configured to receive a bone screw therethrough. Location of the superior screw aperture and the inferior screw aperture near the anterior ends of their respective endplates and the relative disposition of the superior screw aperture and the inferior screw aperture may provide for ease of fixation of the endplates to anatomy, such as to the first and second vertebrae.

The superior endplate may define solely one superior screw aperture and the one superior screw aperture may be located substantially midway across the superior endplate in the transverse direction. The inferior endplate may define solely two inferior screw apertures which may be spaced apart in the transverse direction. The two inferior screw apertures may be located substantially a same distance from midway on the inferior endplate in the transverse direction. The disposition of the superior screw aperture and the two inferior screw apertures may provide for balanced fixation of endplates to their respective vertebrae and such that risk of rotation of endplates may be reduced and distribution of force across the endplates may be maintained.

The core component and the endplate may be configured such that transverse sides of the core component are received between and are adjacent the first and second fingers during the first stage of insertion of the core component. Movement of the core component relative to the endplate may thus be guided during insertion of the core component. The posterior end of the core component may bear against the first and second protrusions towards the end of the first stage of insertion and immediately before the second stage of insertion. The core component may thus be guided from its initial insertion until the core component is nearly in registration with the endplate whereupon the first and second arms are flexed prior to reception of the first and second protrusions in their respective recesses.

Further embodiments of the first aspect of the present invention may comprise one or more features of any aspect of the present invention described below.

According to a second aspect of the present invention there is provided a method of installing an intervertebral fusion device in an intervertebral space between first and second adjacent vertebrae, the intervertebral fusion device comprising at least one endplate, a core component, first and second fingers and first and second recesses, the method comprising:
bringing the core component into engagement with the endplate by moving the core component relative to the endplate; and
inserting the intervertebral fusion device into the intervertebral space, wherein
a proximal end of each of the first and second fingers is attached to the endplate at or adjacent an anterior end of the endplate that first receives the core component when the core component is brought into engagement with the endplate,
each of the first and second fingers extends in a direction of insertion of the intervertebral fusion device into the intervertebral space towards a posterior end of the endplate and is unattached along its length except at its proximal end whereby the finger flexes along its length,
the first and second fingers are spaced apart from each other in a transverse direction which is orthogonal to the direction of insertion and to a direction of separation of the first and second vertebrae,
the first finger defines a first protrusion at a distal end thereof and the second finger defines a second protrusion at a distal end thereof, each protrusion protruding from the respective finger in the transverse direction,
the core component defines the first and second recesses near a posterior end of the core component, the first and second recesses spaced apart from each other in the transverse direction, and
the core component bears against the first and second protrusions during a first stage of engagement of the core component with the endplate to flex the first and second fingers in the transverse direction, and during a second stage of engagement after the first stage of engagement the first and second protrusions are received in the first and second recesses respectively and under spring bias exerted by the first and second fingers to thereby retain the core component relative to the endplate.

According to the method, the core component is brought into engagement with the endplate by moving the core component relative to the endplate. Also, the intervertebral fusion device is inserted into the intervertebral space. In a first approach, the endplate may be positioned in the intervertebral space and then the core component may be brought into engagement with the endplate. In a second approach, the core component may be brought into engagement with the endplate and then the engaged core component and endplate may be inserted together into the intervertebral space.

Further embodiments of the second aspect of the present invention may comprise one or more features of the first aspect of the present invention.

According to a third aspect of the present invention there is provided an oblique lateral intervertebral fusion device, comprising:
at least one endplate configured to be received in an intervertebral space defined between first and second vertebrae;
a core component configured to engage with the at least one endplate when the at least one endplate is in the intervertebral space and the core component is inserted into the intervertebral space;
first and second fingers; and
first and second recesses, wherein
proximal ends of the first and second fingers are attached to the endplate at first and second attachment locations respectively and towards an anterior end of the endplate that first receives the core component when the core component is inserted into the intervertebral space,
each of the first and second fingers extends in the direction of insertion of the core component towards a posterior end of the endplate and is unattached along its length except at its proximal end whereby the finger flexes along its length,
the first and second fingers are spaced apart from each other in a transverse direction which is orthogonal to a direction of insertion of the core component and to a direction of separation of the first and second vertebrae,
the first finger defines a first protrusion and the second finger defines a second protrusion, each protrusion protruding from the respective finger in the transverse direction,
the core component defines the first and second recesses, the first and second recesses spaced apart from each other in the transverse direction,
the core component bears against the first and second protrusions during a first stage of insertion to flex the first and second fingers in the transverse direction, and during a second stage of insertion after the first stage of insertion the first and second protrusions are received in the first and second recesses respectively and under spring bias exerted by the first and second fingers to thereby retain the core component relative to the endplate, and
the first attachment location is spaced further in the direction of insertion from the anterior end of the endplate than the second attachment location.

The present aspect is directed to an oblique lateral intervertebral fusion device, i.e. intervertebral fusion device which is inserted into the intervertebral in the oblique lateral direction. The proximal ends of the first and second fingers are attached to the endplate at first and second attachment locations respectively and towards the anterior end of the endplate that first receives the core component when the core component is inserted into the intervertebral space. The first attachment location is spaced further in the direction of insertion from the anterior end of the endplate than the second attachment location. The first attachment location may be spaced further solely in the direction of insertion of: the direction of insertion and the transverse direction. Spacing of the first and second attachment locations may thus be determined in respect of distance in the direction of insertion only and not distance in the transverse direction. Having the first and second attachment locations relatively disposed in this fashion may provide for configuration of the intervertebral fusion device for oblique lateral insertion and for engagement of the core component with the endplate that is advantageous for oblique lateral insertion.

The anterior end of the endplate may not lie substantially in the same plane. For example, the anterior end of the endplate may have an arcuate or nonlinear profile. The first attachment location may therefore be spaced further than the second attachment location from an anteriormost location at the anterior end of the endplate and in the direction of insertion.

An unattached length of the first arm may be substantially the same as an unattached length of the second arm. The first and second arms may therefore provide for balanced guidance of the core component during insertion of the core component. Furthermore, the first attachment location may be spaced further in the direction of insertion from the anterior end than the second attachment location by at least 10% and more specifically by at least 20% of the unattached length of the first arm.

The core component and the endplate may be configured such that transverse sides of the core component are received between and are adjacent the first and second fingers during the first stage of insertion of the core component to thereby guide movement of the core component relative to the endplate. Furthermore, the core component may define a first posterior corner between a posterior end and a first transverse side of the core component and a second posterior corner between the posterior end and a second transverse side of the core component. The core component may be generally rhomboidal when in plan view whereby the first posterior corner engages with the first arm at substantially the same time as the second posterior corner engages with the second arm upon insertion of the core component. The core component may thus be shaped for oblique lateral insertion. Furthermore, engagement of the first and second posterior corners with their respective arms at substantially the same time may provide for proper orientation of the core component relative to the endplate upon first engagement of the core component with the endplate.

Further embodiments of the third aspect of the present invention may comprise one or more features of the first aspect of the present invention or of any aspect of the invention described below.

According to a further aspect of the present invention there is provided an intervertebral fusion device comprising:
a superior component having a superior component top side and a superior component bottom side, the superior component being configured to be received in an intervertebral space between first and second vertebrae whereby the superior component top side abuts against the first vertebra;
an inferior component having an inferior component top side and an inferior component bottom side, the inferior component being configured to be received in the intervertebral space between the first and second vertebrae whereby the inferior component bottom side abuts against the second vertebra, the superior component bottom side and the inferior component top side opposing each other when the superior and inferior components are received in the intervertebral space;
a core component configured for insertion between the superior and inferior components whereby a separation between the superior and inferior components is determined; and
first and second retention mechanisms operative to resist ejection of the core component from between the superior and inferior components,
wherein each of the first and second retention mechanisms comprises a first portion and a second portion, one of the first and second portions being unitary with one of the superior and inferior components and the other of the first and second portions being unitary with the core component,
wherein the first portion comprises a first inter-engaging formation which is urged in an inter-engaging direction orthogonal to each of a direction of insertion of the core component between the superior and inferior components and a direction of separation of the superior and inferior components, the inter-engaging direction of the first retention mechanism being opposite the inter-engaging direction of the second retention mechanism, and
wherein the second portion comprises a second inter-engaging formation, the first inter-engaging formation being urged to inter-engage with the second inter-engaging formation upon insertion of the core component between the superior and inferior components.

The intervertebral fusion device comprises three main components, namely a superior component (which constitutes a first or superior endplate), an inferior component (which constitutes a second or inferior endplate) and a core component. In use, the superior and inferior components are placed in an intervertebral space between first and second vertebrae formed by at least partial removal of a problematic intervertebral disc. The superior component has a superior component top side and a superior component bottom side with the superior component being placed in the intervertebral space such that the superior component top side faces the first vertebra or what might remain of a partially removed intervertebral disc. The inferior component has an inferior component top side and an inferior component bottom side with the inferior component being placed in the intervertebral space such that the inferior component bottom side faces the second vertebra or what might remain of a partially removed intervertebral disc. The superior component bottom side and the inferior component top side oppose each other when the superior and inferior components are received in the intervertebral space. The superior and inferior components may be in registration with each other when in the intervertebral space and more specifically when the core component is fully inserted between the superior and inferior components as described below.

The core component is configured for insertion between the superior and inferior components. In use, the core component may be inserted between the superior and inferior components when the superior and inferior components have been placed in the intervertebral space, as described above. Upon insertion the core component determines a separation between the superior and inferior components and hence a height of the intervertebral fusion device with the superior component top side abutting against the first vertebra or what remains of the partially removed intervertebral disc and with the inferior component bottom side abutting against the second vertebra or what remains of the partially removed intervertebral disc. Differing heights of intervertebral fusion device may be provided by selection from plural core components of different height.

As mentioned above, an intervertebral disc functions as a shock absorber. An intervertebral disc is therefore under load with the pattern of loading varying and repeating over an extended period. When installed in place of the intervertebral disc, the intervertebral fusion device is likewise under load with such loading being liable to eject the core component from between the superior and inferior components. Usually, such loading provides for ejection to an inconsiderable extent at any one time. Nevertheless, such fractional ejection can become appreciable over time. Ejection is a risk for a core component of any shape, such as a core component having substantially parallel upper and lower surfaces, and not just a risk for the wedge-shaped core component described below. The intervertebral fusion device therefore further comprises first and second retention mechanisms. The first and second retention mechanisms are operative to resist ejection of the core component from between the superior and inferior components.

Each of the first and second retention mechanisms comprises a first portion and a second portion. One of the first and second portions is unitary with one of the superior and inferior components. The superior or inferior component may be formed by the like of casting, moulding or printing whereby one of the first and second portions is integrally formed with the superior or inferior component. Alternatively, the superior or inferior component may be formed by the like of machining or stamping and one of the first and second portions may be formed separately from the superior or inferior component by the like of machining or stamping. The thus formed separate portion may then be attached to the thus formed superior or inferior component by welding, a press fit mechanism or the like whereby the portion is unitary with the superior or inferior component.

In a form, the first portion may be unitary with one of the superior and inferior components. Having the first portion on the superior component or the inferior component in preference to the core component may minimise the amount of material used in the core component and thereby afford an aperture of greater extent in the core component for admitting bone graft material to the interior of the spinal fusion device.

Loading of the intervertebral fusion device is usually from above the superior component. If the superior and inferior components are both gripped by their adjacent vertebrae, loading from above will tend to eject the core component from between the superior and inferior components. However, if the core component and the inferior component are held firmly together, such loading is more liable to push the superior component down onto the core component than eject the core component. Therefore, in a more specific form of the intervertebral fusion device, the first portion may be unitary with the inferior component.

The first portion comprises a first inter-engaging formation. Furthermore, the first portion is configured to urge the first inter-engaging formation in an inter-engaging direction orthogonal to each of a direction of insertion of the core component between the superior and inferior components and a direction of separation of the superior and inferior components. Each of the first and second retention mechanisms therefore has its respective inter-engaging direction of movement of first inter-engaging formation. More specifically, the inter-engaging direction of the first retention mechanism is opposite the inter-engaging direction of the second retention mechanism. Such a structure involving first and second retention mechanisms working in opposite directions may hold the core component sufficiently to provide adequate resistance to ejection of the core component.

The first and second retention mechanisms may be spaced apart from each other and more specifically may be spaced apart from each other in a direction transverse to a direction of insertion of the core component between the superior and inferior components. More specifically, the first and second retention mechanisms may be spaced apart from each other in a direction orthogonal to each of a direction of insertion of the core component between the superior and inferior components and a direction of separation of the superior and inferior components. The first and second retention mechanisms may be on opposite sides of the core component upon insertion of the core component between the inferior and superior components. Holding the core component at its opposite sides may provide a firm hold on the core component.

The second portion comprises a second inter-engaging formation. The second inter-engaging formation may be immovable in relation to the component, such as the core component, in which it is comprised. The second inter-engaging formation may therefore be a profile, such as a recess, defined on a surface of the component in which the second inter-engaging formation is comprised. As mentioned above, the first portion is configured to urge the first inter-engaging formation in the inter-engaging direction. When so urged and upon insertion of the core component between the superior and inferior components, the first inter-engaging formation inter-engages with the second inter-engaging formation. Inter-engagement of the first and second inter-engaging formations of each of the first and second retention mechanisms provides for resistance to ejection of the core component from between the superior and inferior components when the intervertebral fusion device is subject to load and perhaps also when the intervertebral fusion device is subject to load over time.

The intervertebral fusion device may further comprise third and fourth retention mechanisms. Each of the third and fourth retention mechanisms may have a form as described above in respect of the first and second retention mechanisms. The first and second retention mechanisms may be constituted in the core component and the inferior component. The third and fourth retention mechanisms may be constituted in the core component and the superior component. The intervertebral fusion device may thus be configured to provide for resistance to movement of the core component relative to the inferior component and the superior component. It is advantageous to provide for resistance to movement of the core component relative to both the inferior component and the superior component.

Each of the first and second inter-engaging formations may be at a location on a respective one of the inferior or superior component and the core component whereby the first inter-engaging formation inter-engages with the second inter-engaging formation upon insertion of the core component between the inferior and superior components to a predetermined extent at which the first and second inter-engaging formations are in registration with each other. More specifically, the first and second inter-engaging formations may be at locations whereby there is inter-engagement of the first and second inter-engaging formations when the core component is fully inserted between the inferior and superior components.

The core component may have an upper side and a lower side. When the core component is inserted between the inferior and superior components, the upper side may face the superior component bottom side and the lower side may face the inferior component top side. The core component may have first and second lateral sides which each face in a direction orthogonal to a direction of insertion of the core component and to a direction of separation of the inferior and superior components, with the first and second lateral sides facing in opposite directions. An inter-engaging formation, such as a recess, may be located on each of the first and second lateral sides.

The upper side and a lower side of the core component may be inclined to each other. The core component may therefore have the form of a wedge. Furthermore, the upper side and a lower side may not meet at an acute angle whereby the core component has the form of a frustum of a wedge. The core component and the inferior and superior components may be configured for insertion of the thinner edge of the thinner and thicker edges of the core component. An inclination of the inferior and superior components relative to each other may thus be determined by way of the core component further to a separation between the inferior and superior components. Extent of inclination of the inferior and superior components may be determined by selection from a plurality of core components having upper and lower sides of different inclinations. Such selection may be combined with selection from a plurality of core components having different heights. Where the intervertebral fusion device comprises a core component in the form of a wedge, loading of the intervertebral fusion device is more liable to eject the core component from between the inferior and superior components. It may therefore be desirable for the intervertebral fusion device to comprise first to fourth retention mechanisms, as described above, rather than a subset thereof to provide for proper retention of the core component between the inferior and superior components.

The first portion may be configured to urge the first inter-engaging formation in the inter-engaging direction by way of a spring bias. The first portion may therefore further comprise a sprung portion which urges the first inter-engaging formation in the inter-engaging direction. The sprung portion may be a cantilever spring. The sprung portion may be straight or tapered. Tapering controls stiffness and hence extent of deflection. A cantilever sprung structure may be simpler and more compact than other means of providing spring bias. A more compact structure is desirable in an intervertebral fusion device in which space is usually at a premium. As described below, the cantilever sprung structure may lie along a direction of insertion of the core component between the superior and inferior components whereby little space is occupied by the cantilever sprung structure in the transverse direction.

The first inter-engaging formation may be spaced apart on the cantilever spring from a proximal end of the cantilever spring. The first inter-engaging formation may comprise a protrusion. The second inter-engaging formation may comprise a recess with the recess and protrusion being configured for reception of the protrusion in the recess to thereby provide for inter-engagement. The first portion may have the form of a finger having the first inter-engaging formation towards a distal end of the finger. The first portion may extend in a direction of insertion of the core component between the inferior and superior components. A proximal end of the first portion may be located towards an edge of an inferior or superior component that first receives the core component when the core component is inserted. Having the proximal end located towards the core component receiving edge means that the first portion may be less liable to damage when the core component is inserted between the inferior and superior components. As mentioned above, the first portion is unitary with one of the inferior or super components and the core component and, more specifically, may be unitary with one of the inferior or super components in a form of the intervertebral fusion device. The component with which the first portion is unitary may be formed of a material, such as one of the materials described below, of sufficient yield strength to provide the spring bias.

As mentioned above, the first inter-engaging formation may comprise a protrusion and the second inter-engaging formation may comprise a recess with the recess and protrusion being configured for reception of the protrusion in the recess to thereby provide for inter-engagement. Reception of the protrusion in the recess may be perceived by the surgeon, such as by way of an audible click or a reduction in force as the core component advances, whereby the surgeon is notified that the intervertebral fusion device is properly assembled. Furthermore, this structure may allow for trial insertion of a core component or several different core components in turn but without full insertion to the extent of inter-engagement to assess degree of spinal alignment, for example before committing to full insertion of a selected core component. Assessment of such trial insertion may be assisted by a known imaging technique.

At least one of the protrusion and the recess may have a sloping side whereby the protrusion rides down the sloping side as extent of insertion of the core component increases. Furthermore, configuration of the first portion to urge the protrusion in the inter-engaging direction by way of the spring bias may draw the core component between the superior and inferior components under the force of the spring bias. The core component may thus be fully inserted between the superior and inferior components.

Where the first inter-engaging formation is comprised in one of the superior and inferior components, the first inter-engaging formation may be at a first distance from an edge of the inferior or superior component that first receives the core component when the core component is inserted. Furthermore, and where the second inter-engaging formation is comprised in the core component, the second inter-engaging formation may be at a second distance from an edge of the core component opposite an edge that is first received between the superior and inferior components, the first distance being greater than the second distance. Such a structure may provide for the core component being pulled fully between the superior and inferior components by way of the spring bias described above. Furthermore, and where the core component and the inferior or superior component comprise respective inter-engaging anterior formations, the inter-engaging anterior formations may be brought into inter-engagement by the spring bias and by virtue of the first distance being greater than the second distance.

The inter-engaging anterior formations may be configured to draw the superior or inferior component and the core component together. The inter-engaging anterior formations may therefore comprise cooperating surface profiles that cooperate to move the superior or inferior component and the core component together as an extent of insertion of the core component between the superior and inferior components increases. At least one of the inter-engaging anterior formation on the core component and the inter-engaging anterior formation on the superior or inferior component may be sloped with a direction of the slope being such that the superior or inferior component and the core component are drawn together. The spring bias exerted by the first inter-engaging formation may therefore provide force to draw the core component between the superior and inferior components and also to draw the superior or inferior component and the core component together. In a form of the device, the inter-engaging anterior formation may be on the superior component whereby the superior component is drawn down onto the core component.

The device may comprise a further pair of inter-engaging anterior formations. A first one in the further pair of inter-engaging anterior formations may be comprised in the inferior component and a second one in the further pair may be comprised in the core component. The further pair of inter-engaging anterior formations may be configured to resist ejection of the core component from between the superior and inferior components. The first inter-engaging anterior formation may have the form of a recess and the second inter-engaging anterior formation may comprise a protrusion which is received in the recess to present resistance to ejection. The second inter-engaging anterior formation may further comprise a cantilever spring on which the protrusion is defined, the cantilever spring urging the protrusion in a direction of separation of the superior and inferior components whereby the protrusion is urged into the recess of the first inter-engaging anterior formation.

The core component and the respective one of the inferior and superior components may be configured in respect of their relative dimensions to move the first inter-engaging formation against its spring bias at the end of a first stage of insertion of the core component between the inferior and superior components. More specifically, and where the first inter-engaging formation is comprised in one of the superior and inferior components, a leading edge of the core component may be bevelled whereby the first inter-engaging formation is progressively deflected against the spring bias as the first inter-engaging formation rides up the bevelled edge. Energy may thus be stored in the first portion to provide for urging of the first inter-engaging formation towards inter-engagement. The first and second inter-engaging formations may be brought into inter-engagement by way of the spring bias during a second stage of insertion following the first stage of insertion.

As described above, the first portion may extend in a direction of insertion of the core component between the inferior and superior components. The first portions of the first and second retention mechanisms may extend in substantially parallel directions. Where the first portions are comprised in the inferior or superior component, the first portions may be located towards lateral sides of the inferior or superior component.

The core component and at least one of the inferior component and superior component may comprise respective inter-engaging posterior formations. Where an inter-engaging posterior formation is comprised in the superior component, the inter-engaging posterior formation of the superior component and the inter-engaging posterior formation of the core component may be configured to draw the superior component and the core component together. The inter-engaging posterior formations may therefore comprise cooperating surface profiles, with, for example, at least one surface profile being sloped, that cooperate to move the superior component and the core component together as an extent of insertion of the core component between the superior and inferior components increases. The spring bias exerted by the first inter-engaging formation, which is described above, may provide force to cause cooperation between the surface profiles and draw the superior component and the core component together. In addition, a distal edge of an inter-engaging posterior formation, and more specifically the distal edge of the inter-engaging posterior formation on the core component, may be configured to deflect when cooperating with the other inter-engaging posterior formation with inherent spring bias of the material of the deflecting inter-engaging posterior formation exerting force to draw the superior component and the core component together.

The superior component, the inferior component and the core component may be separate components. Having separate inferior and superior components and core component means that the components may be introduced to the intervertebral space more gently compared with known single piece intervertebral fusion devices which often need to be hammered into place. Such a less gentle insertion process may damage the intervertebral fusion device, may increase time required for the intervertebral fusion device to settle in the intervertebral space, and may result in trauma to vertebral bodies, adjacent soft tissues including neural structures. On the subject of trauma, a device that is hammered into place is liable to create microfractures in the vertebrae which could lead to subsidence of the device into the host bone. Furthermore, having separate components and in particular a core component separate to the inferior and superior components allows for differences in dimensions of intervertebral spaces, differences in angle between the adjacent vertebrae that define the intervertebral space, and degree of spinal alignment and/or correction. Each of the superior component, the inferior component and the core component may be integrally formed. The superior component and the inferior component may not engage with each other, other than by way of the core component.

Each of the inferior and superior components may have the form of a plate, albeit a plate having structures thereon that provide for mechanical engagement with the core component, whereby it is thin relative to its length and width. At least one of the superior component top side and the inferior component bottom side may be shaped in the coronal or sagittal planes, for example domed, to enhance fit and contact with the adjacent vertebrae.

References herein to anterior or to anterior aspect are to the anterior aspect of the intervertebral fusion device itself and not to the anterior aspect of the patient. The anterior aspect of the intervertebral fusion device itself therefore means the aspect at which the core component is inserted between the superior and inferior components. Correspondingly, references herein to posterior or to posterior aspect are to the posterior aspect of the intervertebral fusion device itself and not to the posterior aspect of the patient. The anterior and posterior aspects are oppositely directed. The intervertebral fusion device may be an anterior, anterior oblique, lateral or direct lateral intervertebral fusion device.

At least one of the superior component top side and the inferior component bottom side may be configured to provide for fusion. For example, the top or bottom side may comprise formations, such as protrusions, which, in use, engage with the bone of the vertebra. By way of another example, the top and/or bottom side may define apertures for passage of bone graft material therethrough from an interior of the intervertebral fusion device. By way of a further example, the top or bottom side may have a coating thereon or impregnation therein. The coating or impregnation may comprise material that provides for bone adhesion and/or bone formation to encourage bone to grow up to and bond onto the intervertebral fusion device to thereby provide long term stable attachment. One or more known coatings may be used, such as porous mesh, tricalcium phosphate (TCP), hydroxyapatite (HA) or bone morphogenetic protein (BMP).

At least one of the superior component, the core component and the inferior component may be formed from a metal, such as titanium, or a metal alloy, such as stainless steel, Ti6Al4V, CoCr or nitinol. Nitinol may be useful in respect of cooperating parts of the superior component, the core component and the inferior component. At least one of the superior component, the core component and the inferior component may be formed from a plastics material and more specifically a thermoplastic polymer, such as PEEK or carbon reinforced PEEK. In forms of the invention, the core component may be formed by 3D printing whereby the core component has the form of a 3D lattice. The aforementioned materials may be used to form the core component by way of 3D printing.

When assembled, the intervertebral fusion device may have a range of length by width from 20 mm by 15 mm to 65 mm by 50 mm. Where there is an oblique intervertebral fusion device, the range of length by width may be from 20 mm by 15 mm to 40 mm by 35 mm. Where there is an anterior intervertebral fusion device, the range of length by width may be from 20 mm by 20 mm to 50 mm by 50 mm. Where there is a lateral intervertebral fusion device, the range of length by width may be from 40 mm by 18 mm to 65 mm by 40 mm. A height of the intervertebral fusion device may be 5 mm to 15 mm at the posterior aspect.

According to a yet further aspect of the present invention there is provided a method of installing an intervertebral fusion device in an intervertebral space between first and second adjacent vertebrae, the intervertebral fusion device comprising a superior component having a superior component top side and a superior component bottom side, an inferior component having an inferior component top side and an inferior component bottom side, and a core component, the method comprising:
positioning the superior component and the inferior component relative to each other such that the superior component bottom side and the inferior component top side oppose each other;
inserting the core component between the superior and inferior components whereby a separation between the superior and inferior components is determined; and
disposing the intervertebral fusion device in the intervertebral space such that the superior component top side abuts against the first vertebra and the inferior component bottom side abuts against the second vertebra,
wherein the intervertebral fusion device comprises first and second retention mechanisms operative to resist ejection of the core component from between the superior and inferior components, each of the first and second retention mechanisms comprising a first portion and a second portion, one of the first and second portions being unitary with one of the superior and inferior components and the other of the first and second portions being unitary with the core component,
wherein the first portion comprises a first inter-engaging formation which is urged in an inter-engaging direction orthogonal to each of a direction of insertion of the core component between the superior and inferior components and a direction of separation of the superior and inferior components, the inter-engaging direction of the first retention mechanism being opposite the inter-engaging direction of the second retention mechanism, and
wherein the second portion comprises a second inter-engaging formation, the first inter-engaging formation being urged to inter-engage with the second inter-engaging formation upon insertion of the core component between the superior and inferior components.

The intervertebral fusion device may be installed in an intervertebral space by positioning the superior component and the inferior component relative to each other in the intervertebral space before the core component is inserted between the superior and inferior components. Alternatively, the intervertebral fusion device may be installed in an intervertebral space by positioning the superior component and the inferior component relative to each other at a location apart from the intervertebral space and inserting the core component between the superior and inferior components at this location before the thus assembled intervertebral fusion device is installed in the intervertebral space.

Further embodiments of the yet further aspect of the present invention may comprise one or more features of the further aspect of the present invention.

### BRIEF DESCRIPTION OF DRAWINGS

The present invention will now be described by way of example only with reference to the following drawings, of which:

FIG. 1A shows a superior component of a first embodiment of the present invention;

FIG. 1B shows an inferior component of the first embodiment of the present invention;

FIG. 1C shows core component of the first embodiment of the present invention;

FIG. 2A shows the core component before insertion between the superior and inferior components according to a second embodiment;

FIG. 2B shows the core component after insertion between the superior and inferior components according to the second embodiment;

FIG. 3A shows a superior component of a third embodiment of the present invention;

FIG. 3B shows an inferior component of the third embodiment of the present invention;

FIG. 3C shows core component of the third embodiment of the present invention;

FIG. 3D is a view from below of the superior component of FIG. 3A;

FIG. 3E is a view from above of the inferior component of FIG. 3B;

FIG. 3F is a view from above of the core component of FIG. 30;

FIG. 4A shows the core component before insertion between the superior and inferior components according to the third embodiment;

FIG. 4B shows the core component after insertion between the superior and inferior components according to the third embodiment;

FIG. 5A is a view from below of a superior component of a fourth embodiment of the present invention;

FIG. 5B is a view from above a core component of the fourth embodiment;

FIG. 5C is a perspective view of the core component of FIG. 5B;

FIG. 5D shows the fourth embodiment before engagement of the core component with the superior component; and

FIG. 5E shows the fourth embodiment after engagement of the core component with the superior component.

### DESCRIPTION OF EMBODIMENTS

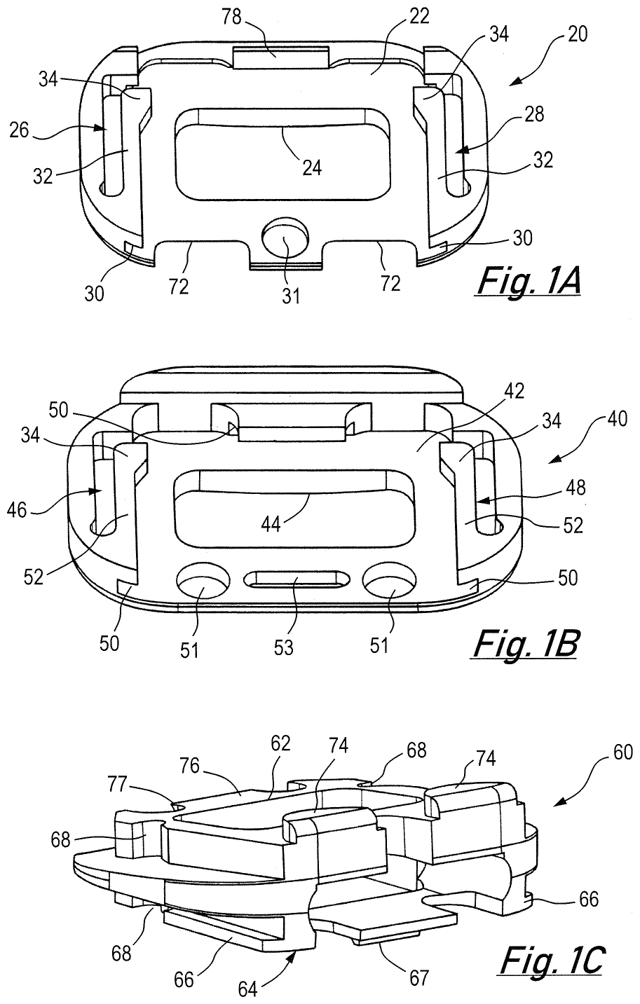

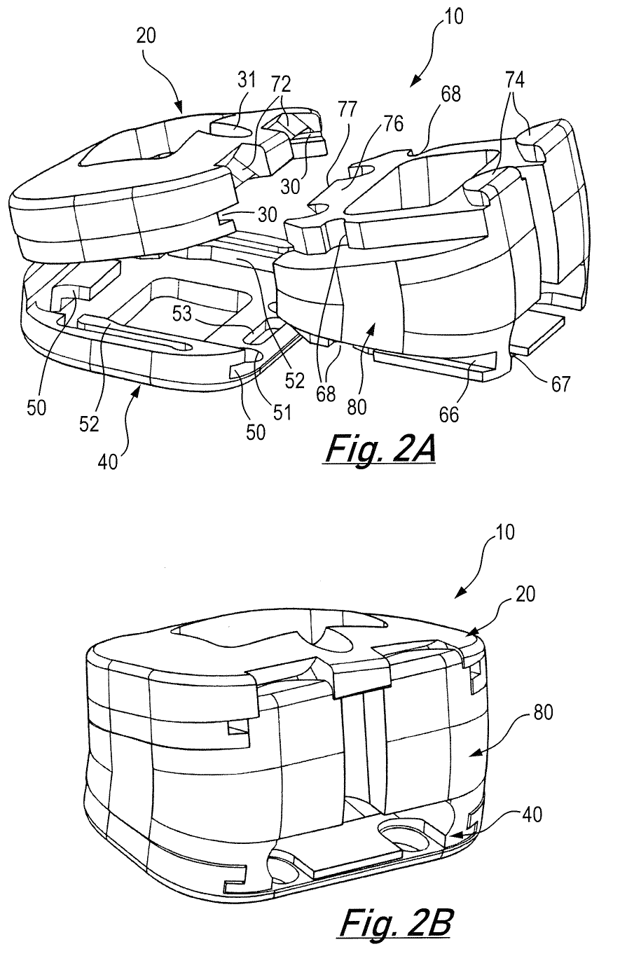

A superior component, an inferior component and a core component of a first embodiment of intervertebral fusion device 10 are shown respectively in FIGS. 1A, 1B and 1C. The superior component constitutes a superior endplate of the intervertebral fusion device 10 and the inferior component constitutes an inferior endplate of the intervertebral fusion device 10. The intervertebral fusion device 10 is an anterior lumbar interbody fusion (ALIF) device. FIG. 2A shows a core component before insertion between the superior and inferior components of FIGS. 1A and 1B according to a second embodiment of intervertebral fusion device with the second embodiment differing from the first embodiment in respect of the greater height of the core component of the second embodiment. FIG. 2B shows the core component of FIG. 2A after insertion between the superior and inferior components.

As mentioned above, the intervertebral fusion device 10 of FIGS. 1A to 2B comprises a superior component 20, an inferior component 40 and a core component 60, 80. Each of the superior component 20 and the inferior component 40 is generally of the form of a plate, albeit a plate having structures thereon and a large aperture through the centre thereof. The core component 60, 80 has the form of a frustum of a wedge. As can be seen from comparison of FIG. 1C with FIGS. 2A and 2B, the core component of FIG. 1C is thinner than the core component of FIGS. 2A and 2B. What is shown in FIGS. 2A and 2B therefore constitutes a second embodiment. Use of core components of different thicknesses and/or different extents of tapering wedge and with the same superior component 20 and inferior component 40 provides for different heights and angles of intervertebral fusion device 10. When the intervertebral fusion device 10 is being brought into use, the superior component 20 and the inferior component 40 are placed in the intervertebral space. It should be noted that the superior component 20 is shown in FIG. 1A upside down so that the features of the underside of the superior component can be seen. In use, the superior component 20 of FIG. 1A is turned upside down when inserted into the intervertebral space. The core component 60, 80 is positioned relative to the superior component 20 and the inferior component 40 as shown in FIG. 2A. Then the core component 60, 80 is positioned between edges of the superior component 20 and the inferior component 40 with the thin edge of the wedge shape of the core component foremost before the core component is progressively inserted between the superior component and the inferior component until fully received between the superior component and the inferior component. FIG. 2B shows the intervertebral fusion device 10 when the core component 60, 80 is fully received between the superior component 20 and the inferior component 40. When in the disposition shown in FIG. 2B, the superior component top side abuts against a first vertebra defining the intervertebral space in part and the inferior component bottom side abuts against a second vertebra defining the intervertebral space in part.

According to an alternative approach to use of the intervertebral fusion device 10, the intervertebral fusion device 10 is assembled outside the intervertebral space, as described above, before the assembled intervertebral fusion device is inserted into the intervertebral space.

The superior component 20 will now be described further with reference to FIG. 1A. The superior component 20 has a superior component bottom side 22, a superior component top side 24, a first lateral side 26 and a second lateral side 28. The superior component 20 comprises various formations 30, 31, 72, 78 on the superior component bottom side 22 which inter-engage with formations on the core component and provide for guided alignment and engagement between core component and superior component during progressive insertion of the core component. The formations 30, 31, 72, 78 also provide for secure attachment of core component and superior component to each other when the core component is fully received in the superior component. The superior component 20 defines a circular aperture 31 towards its anterior edge. The circular aperture 31 allows for reception therethrough of a screw which is driven into the adjacent vertebra. The superior component 20 further comprises two first portions 32 which are integrally formed with the superior component. One of the first portions 32 is located towards the first lateral side 26 and the other of the first portions 32 is located towards the second lateral side 28. Each of the first portions 32 comprises a main body in the form of a finger which extends from a proximal end of the finger at an anterior edge of the superior component across the superior component towards the posterior edge of the superior component. The first portion 32 therefore extends in a direction of insertion of the core component. The first portion 32 is unsupported and attached along its length with the exception of its proximal end. The superior component and hence the first portion 32 are formed of a material of sufficient yield strength that the first portion functions as a cantilever spring. Although not shown in FIG. 1A, the finger is tapered in certain forms to determine stiffness and hence extent of deflection. Furthermore, a radius of the interface between the finger and the part of the superior component from which the finger extents is determined to control stiffness of the finger. The main body of the first portion 32 defines a protrusion 34 (which constitutes a first inter-engaging formation) at its distal end with the protrusion 34 extending in a transverse direction of the superior component. The two first portions 32 are mirror images of each other whereby their two protrusions 34 oppose each other and extend towards each other. In view of the cantilever sprung nature of each first portion 32, application of a load to the protrusion 34 in a transverse direction towards a respective lateral side deflects the main body to thus store energy in the first portion to provide for urging of the protrusion 34 in the opposite direction. As described below, the main body of the finger is deflected by a leading edge of the core component bearing against the protrusion 34.

The inferior component 40 will now be described further with reference to FIG. 1B. The inferior component 40 has an inferior component top side 42, an inferior component bottom side 44, a first lateral side 46 and a second lateral side 48. Like the superior component 20, the inferior component 40 comprises various formations 50 on the inferior component top side 42 which inter-engage with formations on the core component and provide for engagement between core component and inferior component during progressive insertion of the core component. The formations 50 also provide for secure attachment of core component and inferior component to each other when the core component is fully received in the inferior component. However, the form and disposition of the formation 50 at the posterior of the inferior component 40 differs from the form and disposition of the formation 78 at the posterior of the superior component 20. The inferior component 40 defines two circular apertures 51 towards its anterior edge which are spaced apart from each other in the transverse direction. Each circular aperture 51 allows for reception therethrough of a screw which is driven into the adjacent vertebra. The inferior component 40 further comprises two first portions 52 which are integrally formed with the inferior component. One of the first portions 52 is located towards the first lateral side 46 and the other of the first portions 52 is located towards the second lateral side 48. The first portion 52 at the first lateral side 46 of the inferior component is of the same form and function as the first portion 32 at the first lateral side 26 of the superior component. The first portion 52 at the second lateral side 48 of the inferior component is of the same form and function as the first portion 32 at the second lateral side 28 of the superior component. The reader's attention is therefore directed to the preceding description of the two first portions 32 of the superior component 20.

The core component 60 will now be described further with reference to FIG. 1C. As described above, the core component 60 has the form of a frustum of a wedge. The core component 60 has an upper side 62 and a lower side 64, the core component 60 being configured to be inserted between the superior and inferior components 20, 40 such that the upper side 62 faces the superior component bottom side 22 and the lower side 64 faces the inferior component top side 42. This orientation of core component 60 in relation to superior and inferior components 20, 40 is obtained by various formations 66, 67, 74, 76 on the upper and lower sides 62, 64 with the formations on the upper side being of different form and disposition to the formations on the lower side. Furthermore, the formations 66, 67, 74, 76 on each side of the core component correspond to and provide for engagement with a respective one of the formations 30, 31, 72, 78 on the superior component and the formations 50 on the inferior component. The core component 60 defines four recesses 68 having sloping sides (each recess constitutes a second inter-engaging formation). Two of the recesses are located towards the upper side and two of the recesses are located towards the lower side. Furthermore, the recesses are located towards the thin edge of the wedge. Each of the recesses 68 faces in a transverse direction away from the core component.

As described above, the superior and inferior components 20, 40 are placed in the intervertebral space and the core component 60, 80 is positioned relative to the superior and inferior components as shown in FIG. 2A before the core component is inserted between the superior and inferior components. As the leading edge of the core component 60, 80 reaches the protrusions 34 of the superior and inferior components 20, 40 during insertion, the leading edge of the core component bears against each of the protrusions causing deflection of each of the four first portions 32, 52. The part of the leading edge that bears against a respective protrusion is bevelled to provide for increasing deflection as the extent of insertion of the core component is increased. Upon further insertion the protrusions 34 and the recesses 68 come into registration and each of the protrusions is urged under spring bias into a corresponding one of the recesses. Reception of the protrusions in their respective recesses produces an audible click which indicates to the surgeon that the core component is fully inserted. Inter-engagement between protrusions 34 and recesses 68 contributes to holding the core component 60, 80, and superior and inferior components 20, 40 together and more importantly presents resistance to ejection of the core component from between the superior and inferior components during use of the intervertebral fusion device 10.

As mentioned above, each of the recesses 68 on the core component 60 has sloping sides. When the core component is inserted to the extent that a protrusion 34 reaches its respective recess, the spring bias of the finger causes the protrusion to ride down the sloped side of the recess and thereby exert force to draw the core component further between the superior and inferior components. Further to this, the protrusion 34 is at a first distance from an anterior edge of the inferior or superior component and the recess 68 is at a second distance from an anterior edge of the core component with the first distance being slightly greater than the second distance. These relative distances in combination with the spring bias pull the core component fully between the superior and inferior components.

The core component 60 has two anterior formations 74 on its upper side 62 in the form of protrusions which are spaced apart from each other in the transverse direction along the anterior edge of the core component. Each anterior formation 74 has a side oriented towards the posterior edge of the core component which slopes away from a distal surface of the anterior formation towards the anterior edge. The superior component 20 has two recesses 72 which are spaced apart from each other in the transverse direction and which are defined in the anterior edge of the superior component. When the core component is nearly fully inserted between the inferior and superior components, a base of each recess 72 bears against the sloping side of its respective anterior formation 74 with further insertion causing the base to ride along the sloping side. As the base rides along the sloping side, the superior component is drawn down onto the core component. The pulling of the core component fully between the superior and inferior components by the spring bias, as described above, also causes the base to ride along the sloping side whereby the spring bias also draws the superior component down at its anterior edge onto the core component. The first distance being slightly greater than the second distance, as described above, causes the superior component to be drawn closely against the core component.

The core component 60 has a posterior formation 76 in the form of a protrusion which extends from the posterior edge of the core component adjacent the upper side 62 of the core component. An upper surface of the posterior formation 76 is defined by the upper side 62 of the core component. A distal edge of the posterior formation 76 slopes downwards from a distal edge 77 at the upper surface of the posterior formation and towards the posterior edge of the core component to define a slope. The superior component 20 has a posterior formation 78 in the form of a protrusion which extends from the posterior edge of the superior component in a direction opposite the direction of insertion of the core component over the superior component bottom side 22. The posterior formation 78 defines a distal surface which faces towards the inferior component when the inferior and superior components are aligned. A side of the posterior formation 78 oriented towards the anterior edge of the superior component slopes from an edge of the distal surface furthest from the posterior edge upwards and towards the posterior edge. When the core component is nearly fully inserted between the inferior and superior components, the slope defined by the posterior formation 76 of the core component and the slope defined by the posterior formation 78 of the superior component ride over each other to draw the superior component down onto the core component. The pulling of the core component fully between the superior and inferior components by the spring bias, as described above, also causes the slopes defined by the two posterior formations 76, 78 to ride over each other whereby the spring bias also draws the superior component down at its posterior edge onto the core component. The distal edge 77 at the upper surface of the posterior formation 76 of the core component is configured by virtue of the thinness of the distal edge having regard to the material properties of the core component to deflect when cooperating with the posterior formation 78 of the superior component. Inherent spring bias of the material of the posterior formation 76 adjacent the distal edge 77 exerts force to draw the superior component and the core component together to thereby provide a further means of drawing the superior component down at its posterior edge onto the core component.

Referring to FIG. 1C, the core component 60 further comprises in its lower side 64 a living hinge which defines a protrusion 67 thereon. Inherent spring bias of the living hinge urges the protrusion 67 towards the inferior component 40 when the core component 60 is received between the superior and inferior components 20, 40. When the core component 60 is at full insertion, the protrusion 67 on the living hinge at the lower side 64 of the core component is urged by the inherent spring bias of the living hinge into an elongate aperture 53 in the inferior component near the anterior edge of the inferior component. Reception of the protrusion 67 in the elongate aperture 53 presents a barrier to ejection of the core component from between the inferior and superior components.

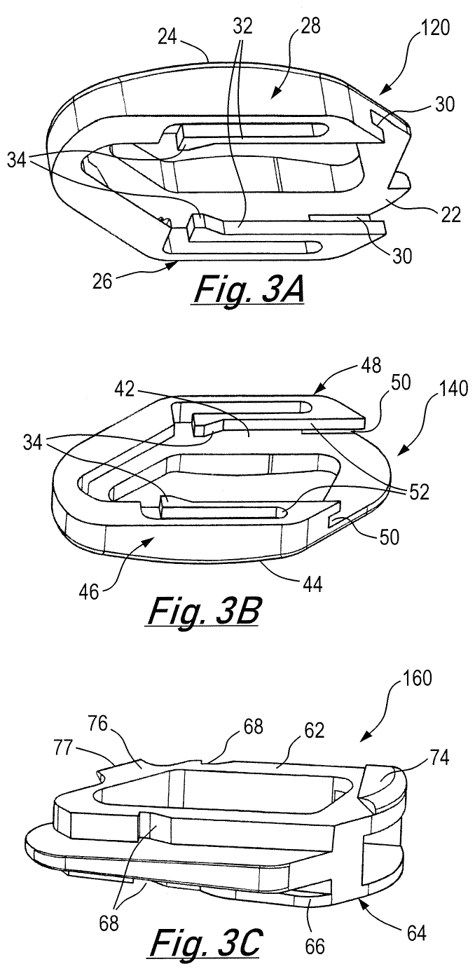

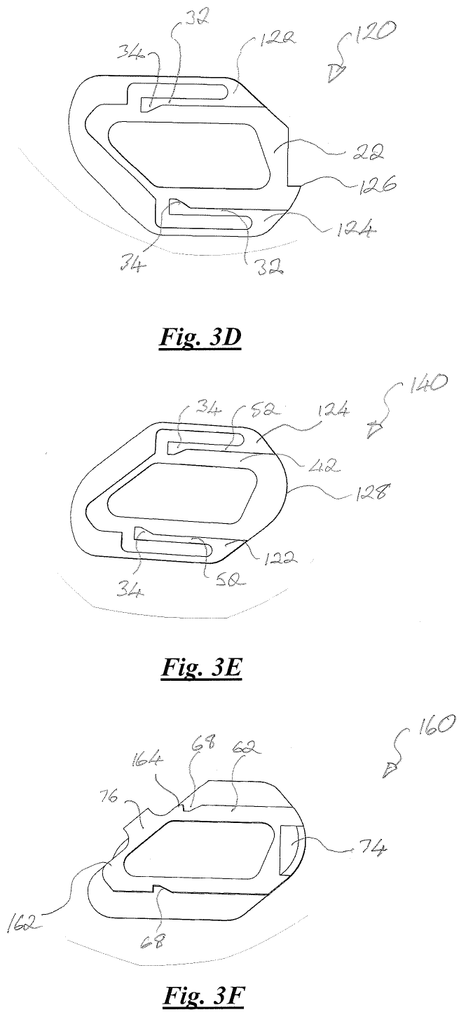

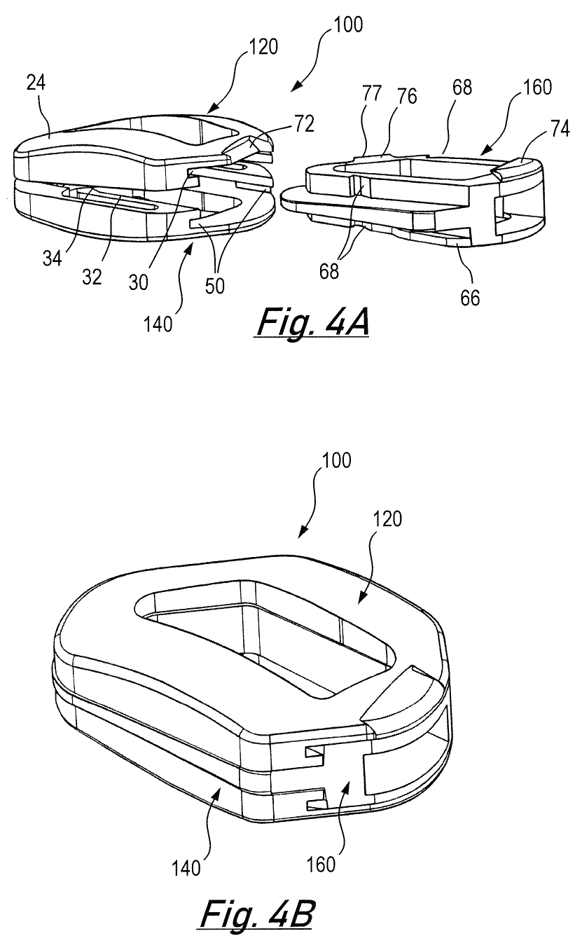

A third embodiment of intervertebral fusion device 100 is shown in FIGS. 3A to 4B. The intervertebral fusion device 100 according to the third embodiment is an oblique lateral interbody fusion device. A superior component 120 (which constitutes a superior endplate), an inferior component 140 (which constitutes an inferior endplate), and a core component 160 of the third embodiment are shown respectively in FIGS. 3A, 3B and 3C and respectively in FIGS. 3D, 3E and 3F. FIG. 4A shows the core component 160 of the third embodiment before insertion between the superior and inferior components 120, 140 of FIGS. 3A and 3B. FIG. 4B shows the core component 160 of the third embodiment after insertion between the superior and inferior components 120, 140 of FIGS. 3A and 3B. The superior component 120, the inferior component 140, and the core component 160 of the third embodiment are of different shape and dimensions to the first and second embodiments when viewed in plan and as shown in FIGS. 3D, 3E and 3F whereby the third embodiment is configured for insertion into an intervertebral space from an oblique lateral direction. Otherwise, and except as described below, the third embodiment is configured in respect of key features in the same fashion as the first and second embodiments. Such key features are therefore indicated in FIGS. 3A to 4B by the same reference numerals as for the first and second embodiments and the reader's attention is directed to the preceding description for a description of such key features.

In respect of differences, as can be seen from FIG. 3C, the core component 160 has one anterior formation 74 on its upper side instead of the two anterior formations 74 of the first and second embodiments. Correspondingly, the superior component 120 of the third embodiment has one recess 72 at its anterior edge instead of the two recesses of the first and second embodiments. Furthermore, the third embodiment lacks the first and second embodiments' combination of the protrusion 67 on the living hinge in the core component and the elongate aperture 53 in the inferior component. Referring to FIGS. 3A and 3D, the two first portions 32 (which constitute first and second arms) of the superior component 120 are attached at their proximal ends towards the anterior end of the superior component. Likewise, and referring to FIGS. 3B and 3E, the two first portions 52 (which constitute third and fourth arms) of the inferior component 140 are attached at their proximal ends towards the anterior end of the inferior component. Each of one of the two first portions 32 of the superior component 120 and one of the two first portions 52 of the inferior component 140 is attached at a first attachment location 122. Each of the other of the two first portions 32 of the superior component 120 and the other of the two first portions 52 of the inferior component 140 is attached at a second attachment location 124. The first attachment location 122 of the superior component 120 is spaced further than the second attachment location 124 of the superior component from an anteriormost location 126 at the anterior end of the superior component and in the direction of insertion of the core component. The first attachment location 122 of the inferior component 140 is spaced further than the second attachment location 124 of the inferior component from an anteriormost location 128 at the anterior end of the inferior component and in the direction of insertion of the core component. The spacing of each of the first and second attachment locations 122, 124 from their respective anteriormost location 126, 128 is in respect of distance in the direction of insertion only and not distance in the transverse direction. Each first attachment location 122 is spaced further in the direction of insertion from the respective anteriormost location 126, 128 than the respective second attachment location 124 by about 30% of the unattached length of the first portion 32, 52.

Referring now to FIG. 3C, the core component 160 is generally rhomboidal in plan view. The rhomboidal form of the core component 160 is more immediately evident from FIG. 3F. The core component 160 defines a first posterior corner 162 on the upper side 62 of the core component between the posterior end and a first transverse side of the core component. The core component 160 further defines a second posterior corner 164 between the posterior end and a second transverse side of the core component. The first and second posterior corners 162, 164 are spaced apart from each other in the direction of insertion by substantially the same distance as the first and second attachment locations 122, 124 are spaced apart whereby the core component has its generally rhomboidal form. The relative position of the first and second posterior corners 162, 164 in the insertion direction results in the first and second posterior corners engaging with their respective first portions 32 at substantially the same time upon first engagement of the core component 160 with the superior component 120. The core component 160 also defines first and second posterior corners on the lower side 64 which correspond to the first and second posterior corners 162, 164 defined on the upper side 62 of the core component. The first and second posterior corners on the lower side 64 of the core component 160 engage with their respective first portions 52 at substantially the same time upon first engagement of the core component 160 with the inferior component 140.

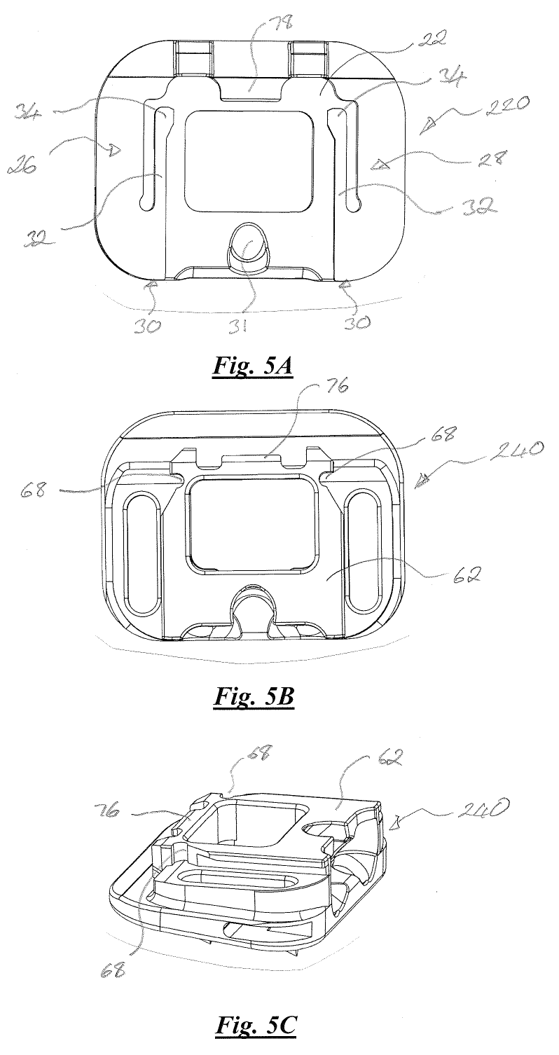

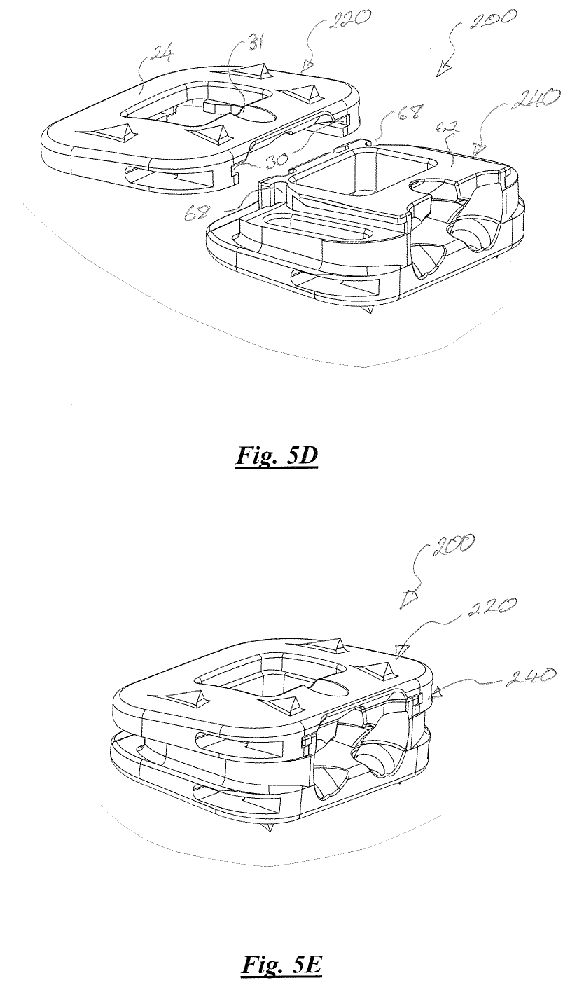

A fourth embodiment of intervertebral fusion device 200 is shown in FIGS. 5A to 5E. The fourth embodiment corresponds to the first embodiment of FIGS. 1A to 2B with the significant exception of the inferior component 40 of the first embodiment being integrally formed with the core component 60 of the first embodiment whereby the inferior component 40 is comprised in the core component 60. The fourth embodiment therefore comprises a superior component 220 and a core component 240.

The superior component 220 of the fourth embodiment is configured in respect of key features in the same fashion as the superior component 20 of the first embodiment. Such key features are therefore indicated in FIG. 5A by the same reference numerals as for the first embodiment and the reader's attention is directed to the preceding description for a description of such key features.

The core component 240 of the fourth embodiment is configured in respect of key features in the same fashion as would result from integral formation of the inferior component 40 of the first embodiment with the core component 60 of the first embodiment. Such key features are therefore indicated in FIGS. 5B and 5C by the same reference numerals as for the first embodiment and the reader's attention is directed to the preceding description for a description of such key features. In view of the inferior component 40 of the first embodiment being integrally formed with the core component 60, structure on the lower side of the core component and structure on the upper side of the inferior component that provides for sliding engagement of the core component with the inferior component is not required in the fourth embodiment whereby it is lacking from the fourth embodiment.

The fourth embodiment 200 is brought into use by positioning the superior component 220 in the intervertebral space and then bringing the core component 240 into sliding engagement with the superior component. As per the first embodiment, movement of the core component 240 relative to the superior component 220 in the direction of insertion of the core component into the intervertebral space and during a first stage of insertion causes the first portions 32 to flex apart whereby the protrusions 34 are pushed further apart. During a second stage of insertion when the core component 240 and the superior component 220 are nearly in registration with each other, the protrusions 34 start to be received in the recesses 68 defined towards the posterior end of the core component. When the core component 240 and the superior component 220 are in registration, the protrusions 34 are properly received in the recesses 68 to thereby present resistance to movement of the core component relative to the superior component in a direction opposite to the direction of insertion. In a different form of use, the superior component 220 and the core component 240 are brought into engagement and registration with each other outside the intervertebral space. The superior component 220 and the core component 240 are then inserted together into the intervertebral space.

1. An intervertebral fusion device, comprising:
at least one endplate configured to be received in an intervertebral space defined between first and second vertebrae;
a core component configured to engage with the at least one endplate when the at least one endplate is in the intervertebral space and the core component is inserted into the intervertebral space;
first and second fingers; and
first and second recesses, wherein
a proximal end of each of the first and second fingers is attached to the endplate at or adjacent an anterior end of the endplate that first receives the core component when the core component is inserted into the intervertebral space,
each of the first and second fingers extends in the direction of insertion of the core component towards a posterior end of the endplate and is unattached along its length except at its proximal end whereby the finger flexes along its length,
the first and second fingers are spaced apart from each other in a transverse direction which is orthogonal to a direction of insertion of the core component and to a direction of separation of the first and second vertebrae,
the first finger defines a first protrusion at a distal end thereof and the second finger defines a second protrusion at a distal end thereof, each protrusion protruding from the respective finger in the transverse direction,
the core component defines the first and second recesses near a posterior end of the core component, the first and second recesses spaced apart from each other in the transverse direction, and
the core component bears against the first and second protrusions during a first stage of insertion to flex the first and second fingers in the transverse direction, and during a second stage of insertion after the first stage of insertion the first and second protrusions are received in the first and second recesses respectively and under spring bias exerted by the first and second fingers to thereby retain the core component relative to the endplate.

2. The intervertebral fusion device according to claim 1, wherein each of the first and second protrusions is located on the endplate and each of the first and second recesses is located on the core component whereby the first and second protrusions are received in the first and second recesses respectively when the core component is substantially fully inserted into the intervertebral space relative to the endplate.

3. The intervertebral fusion device according to claim 1, wherein the intervertebral fusion device comprises first and second endplates and the core component is received between the first and second endplates when the core component is inserted into the intervertebral space.

4. The intervertebral fusion device according to claim 3, wherein the first and second fingers are attached to the first endplate and the intervertebral fusion device comprises third and fourth fingers and third and fourth recesses, the third and fourth fingers attached to the second endplate and the third and fourth recesses defined by the core component, the third and fourth fingers flexing in the transverse direction during the first stage of insertion of the core component between the first and second endplates, and a third protrusion defined by the third finger is received in the third recess and a fourth protrusion defined by the fourth finger is received in the fourth recess during the second stage of insertion of the core component.

5. The intervertebral fusion device according to claim 3, wherein the first endplate is a superior endplate and defines at least one superior screw aperture near the anterior end of the superior endplate and the second endplate is an inferior endplate and defines at least one inferior screw aperture near the anterior end of the inferior endplate, each at least one superior screw aperture at a different location in the transverse direction from each at least one inferior screw aperture when the superior and inferior endplates are in registration, and each at least one superior screw aperture and each at least one inferior screw aperture is configured to receive a bone screw therethrough.

6. The intervertebral fusion device according to claim 5, wherein the superior endplate has a superior component top side and a superior component bottom side, the inferior endplate has an inferior component top side and an inferior component bottom side, wherein the superior component top side faces the first vertebra, the inferior component bottom side faces the second vertebra and the superior component bottom side and the inferior component top side face each other when the superior and inferior endplates are received in the intervertebral space, and wherein each at least one superior screw aperture is substantially circular and extends through the superior endplate from the superior component top side to the superior component bottom side, and each at least one inferior screw aperture is substantially circular and extends through the inferior endplate from the inferior component top side to the inferior component bottom side.

7. The intervertebral fusion device according to claim 5, wherein the superior endplate defines solely one superior screw aperture, the one superior screw aperture located substantially midway across the superior endplate in the transverse direction, and wherein the inferior endplate defines solely two inferior screw apertures which are spaced apart in the transverse direction, the two inferior screw apertures located substantially a same distance from midway on the inferior endplate in the transverse direction.

8. The intervertebral fusion device according to claim 3, wherein the first endplate is a superior endplate and the second endplate is an inferior endplate, the core component has an upper side and a lower side, the upper side facing the superior endplate and the lower side facing the inferior endplate when the core component is inserted between the inferior and superior endplates, the core component defines at least one upper screw aperture near an anterior end and in the upper side of the core component and at least one lower screw aperture near the anterior end and in the lower side of the core component, and each at least one upper screw aperture and each at least one lower screw aperture is configured to receive a bone screw therethrough.

9. The intervertebral fusion device according to claim 8, wherein the superior endplate defines at least one superior screw aperture near the anterior end of the superior endplate and the inferior endplate defines at least one inferior screw aperture near the anterior end of the inferior endplate, each at least one upper screw aperture substantially in registration with a respective one of each at least one superior screw aperture when the superior endplate and core component are in registration, and each at least one lower screw aperture substantially in registration with a respective one of each at least one inferior screw aperture when the inferior endplate and core component are in registration.

10. The intervertebral fusion device according to claim 8, wherein each at least one upper screw aperture is a recess which is open at an anterior end of the core component, and each at least one lower screw aperture is a recess which is open at an anterior end of the core component.

11. The intervertebral fusion device according to claim 3, wherein the first endplate is a superior endplate and the second endplate is an inferior endplate, the core component has an upper side and a lower side, the upper side facing the superior endplate and the lower side facing the inferior endplate when the core component is inserted between the inferior and superior endplate, the core component defining first and second lateral sides which face in opposite transverse directions, the first lateral side extending between the upper and lower sides, the second lateral side extending between the upper and lower sides, at least a part of the first lateral side of the core component is substantially coterminous with a first lateral side of the superior component and a first lateral side of the inferior component, and at least a part of the second lateral side of the core component is substantially coterminous with a second lateral side of the superior component and a second lateral side of the inferior component.

12. The intervertebral fusion device according to claim 1, wherein the first and second protrusions protrude towards each other and the core component and the endplate are configured such that the first and second fingers flex apart in the transverse direction during the first stage of insertion.

13. The intervertebral fusion device according to claim 1, wherein the core component and the endplate are configured such that transverse sides of the core component are received between and are adjacent the first and second fingers during the first stage of insertion of the core component to thereby guide movement of the core component relative to the endplate, and the posterior end of the core component bears against the first and second protrusions towards the end of the first stage of insertion and immediately before the second stage of insertion.

14. The intervertebral fusion device according to claim 1, wherein the intervertebral fusion device comprises first and second endplates, the first and second fingers are attached to the first endplate, and the second endplate is integrally formed with the core component.

15. A method of installing an intervertebral fusion device in an intervertebral space between first and second adjacent vertebrae, the intervertebral fusion device comprising at least one endplate, a core component, first and second fingers and first and second recesses, the method comprising:
bringing the core component into engagement with the endplate by moving the core component relative to the endplate; and
inserting the intervertebral fusion device into the intervertebral space, wherein
a proximal end of each of the first and second fingers is attached to the endplate at or adjacent an anterior end of the endplate that first receives the core component when the core component is brought into engagement with the endplate,
each of the first and second fingers extends in a direction of insertion of the intervertebral fusion device into the intervertebral space towards a posterior end of the endplate and is unattached along its length except at its proximal end whereby the finger flexes along its length,
the first and second fingers are spaced apart from each other in a transverse direction which is orthogonal to the direction of insertion and to a direction of separation of the first and second vertebrae,
the first finger defines a first protrusion at a distal end thereof and the second finger defines a second protrusion at a distal end thereof, each protrusion protruding from the respective finger in the transverse direction,
the core component defines the first and second recesses near a posterior end of the core component, the first and second recesses spaced apart from each other in the transverse direction, and
the core component bears against the first and second protrusions during a first stage of engagement of the core component with the endplate to flex the first and second fingers in the transverse direction, and during a second stage of engagement after the first stage of engagement the first and second protrusions are received in the first and second recesses respectively and under spring bias exerted by the first and second fingers to thereby retain the core component relative to the endplate.

16. An oblique lateral intervertebral fusion device, comprising:
at least one endplate configured to be received in an intervertebral space defined between first and second vertebrae;
a core component configured to engage with the at least one endplate when the at least one endplate is in the intervertebral space and the core component is inserted into the intervertebral space;
first and second fingers; and
first and second recesses, wherein
proximal ends of the first and second fingers are attached to the endplate at first and second attachment locations respectively and towards an anterior end of the endplate that first receives the core component when the core component is inserted into the intervertebral space,
each of the first and second fingers extends in the direction of insertion of the core component towards a posterior end of the endplate and is unattached along its length except at its proximal end whereby the finger flexes along its length,
the first and second fingers are spaced apart from each other in a transverse direction which is orthogonal to a direction of insertion of the core component and to a direction of separation of the first and second vertebrae,
the first finger defines a first protrusion and the second finger defines a second protrusion, each protrusion protruding from the respective finger in the transverse direction,
the core component defines the first and second recesses, the first and second recesses spaced apart from each other in the transverse direction,
the core component bears against the first and second protrusions during a first stage of insertion to flex the first and second fingers in the transverse direction, and during a second stage of insertion after the first stage of insertion the first and second protrusions are received in the first and second recesses respectively and under spring bias exerted by the first and second fingers to thereby retain the core component relative to the endplate, and
the first attachment location is spaced further in the direction of insertion from the anterior end of the endplate than the second attachment location.

17. The oblique lateral intervertebral fusion device according to claim 16, wherein the first attachment location is spaced further in the direction of insertion from an anteriormost location at the anterior end of the endplate than the second attachment location.

18. The oblique lateral intervertebral fusion device according to claim 16, wherein an unattached length of the first arm is substantially the same as an unattached length of the second arm.

19. The oblique lateral intervertebral fusion device according to claim 18, wherein the first attachment location is spaced further in the direction of insertion from the anterior end than the second attachment location by at least 20% of the unattached length of the first arm.

20. The oblique lateral intervertebral fusion device according to claim 16, wherein the core component and the endplate are configured such that transverse sides of the core component are received between and are adjacent the first and second fingers during the first stage of insertion of the core component to thereby guide movement of the core component relative to the endplate.

21. The oblique lateral intervertebral fusion device according to claim 20, wherein the core component defines a first posterior corner between a posterior end and a first transverse side of the core component and a second posterior corner between the posterior end and a second transverse side of the core component, and the core component is generally rhomboidal when in plan view whereby the first posterior corner engages with the first arm at substantially the same time as the second posterior corner engages with the second arm upon insertion of the core component.
